(19) United States
(12) Patent Application Publication
(10) Pub. No.: US 2024/0206794 A1
(43) Pub. Date: Jun. 27, 2024

(54) IN-EAR ELECTROENCEPHALOGRAPHY ELECTRODES WITH MULTI-PARAMETER VITALS MONITOR CONNECTIVITY

(71) Applicants: Ryan Farzam Ahmed, Kew Gardens, NY (US); Shahriar Huda, Jamaica, NY (US); Authoy Das, Jamaica, NY (US); Steffani Rajapaksha, Bayonne, NJ (US); Melani Shrestha, Elmhurst, NY (US); Alisha Karim, Queens, NY (US); Chloe Kan, Oakland Gardens, NY (US); Arun Chhugani, Bethlehem, PA (US); Chengling Zheng, Flushing, NY (US); Genevive Chang, New York City, NY (US); Dang Nguyen, Wellington, FL (US); Pari Patel, Hagerstown, MD (US); Jinnat Mahmood, Jamaica, NY (US); Katherine Xiao, Watkinsville, GA (US); Lesley Osasogielyamu, Mount Vernon, NY (US); Mohammad Rahim, Jamaica, NY (US); Nana Adom Opare-Addo, Bronx, NY (US); Dhriti Rathod, Chambersburg, PA (US); Sakef Uddin, Jamaica, NY (US); Samiha Nasrin, Bronx, NY (US); Saqib Khan, Elmont, NY (US); Saifa Sowa, Hollis, NY (US); Tanvi Jammula, Ashburn, VA (US); Veda Kolli, Ballwin, MO (US); Wajiha Moin, Brooklyn, NY (US); William Yu, Bayside, NY (US); Deven Rozario, Flushing, NY (US)

(72) Inventors: Ryan Farzam Ahmed, Kew Gardens, NY (US); Shahriar Huda, Jamaica, NY (US); Authoy Das, Jamaica, NY (US); Steffani Rajapaksha, Bayonne, NJ (US); Melani Shrestha, Elmhurst, NY (US); Alisha Karim, Queens, NY (US); Chloe Kan, Oakland Gardens, NY (US); Arun Chhugani, Bethlehem, PA (US); Chengling Zheng, Flushing, NY (US); Genevive Chang, New York City, NY (US); Dang Nguyen, Wellington, FL (US); Pari Patel, Hagerstown, MD (US); Jinnat Mahmood, Jamaica, NY (US); Katherine Xiao, Watkinsville, GA (US); Lesley Osasogielyamu, Mount Vernon, NY (US); Mohammad Rahim, Jamaica, NY (US); Nana Adom Opare-Addo, Bronx, NY (US); Dhriti Rathod, Chambersburg, PA (US); Sakef Uddin, Jamaica, NY (US); Samiha Nasrin, Bronx, NY (US); Saqib Khan, Elmont, NY (US); Saifa Sowa, Hollis, NY (US); Tanvi Jammula, Ashburn, VA (US); Veda Kolli, Ballwin, MO (US); Wajiha Moin, Brooklyn, NY (US); William Yu, Bayside, NY (US); Deven Rozario, Flushing, NY (US)

(21) Appl. No.: 18/452,561

(22) Filed: Aug. 21, 2023

(51) Int. Cl. A61B 5/291; A61B 5/00; A61B 5/384

(52) U.S. Cl. CPC A61B 5/291; A61B 5/0006; A61B 5/384; A61B 5/6817; A61B 2505/05

(57) ABSTRACT

The present invention displays an in-the-ear Electroencephalography (EEG) electrode system designed to monitor brain activity in clinical environments, using eartips composed of n-doped silicon or alternative conductive rubber-based materials positioned in the ear canal to accurately detect neurological signals. This electrode system offers wireless and wired configurations, ensuring adaptability to various platforms. This invention addresses the limitations of traditional EEG methodologies by providing a solution for continuous and reliable brain activity monitoring, particularly during intricate surgical procedures. The electrode system displays patient vitals and EEG data in real time, providing medical professionals in diverse clinical settings with comprehensive insights into brain states across a spectrum of frequencies. This system captures Event-Related Potentials (ERPs) and activities in the temporal lobes. Its design minimizes artifacts from facial and scalp muscles and uses advanced signal processing techniques to filter out non-cerebral electrical activities, and its ambulatory usability ensures effectiveness in dynamic scenarios.

### FIELD OF THE INVENTION

The present invention pertains to the field of medical monitoring, specifically to an advanced Electroencephalography (EEG) system. It relates to an in-the-ear EEG electrode system devised for neurological signal detection in operating rooms and clinical settings. This system offers an alternative to traditional scalp EEGs and invasive methods, presenting a method for continuous brain activity monitoring during various medical procedures, including intracranial operations.

### BACKGROUND

Description of the Problem: Electroencephalography (EEG) technology is crucial when monitoring vital signs during neurosurgery and other intricate clinical procedures. However, existing EEG methodologies are limited in their scope in the effective monitoring of brain activity. Conventional scalp EEG electrodes, while minimally invasive, are difficult to use during intracranial operations, unable to account for individual anatomical differences, and require complex setups.

Description of the Related Art: Various techniques and systems have been developed to address the challenge of monitoring brain activity in clinical environments. Conventional scalp EEG electrodes are widely used for non-invasive monitoring but are limited in their adaptability to certain scenarios, such as intracranial procedures. Other solutions include invasive EEG methods that require the insertion of electrodes directly into the brain tissue which introduce serious risks to the patient's safety.

Previous inventions have attempted to combine EEG monitoring with other functionalities, such as audio delivery through earbuds. However, these solutions often have technical limitations such as inadequate signal quality and limited compatibility with existing clinical monitoring platforms. Therefore, a need remains for a comprehensive EEG electrode system that addresses the challenges posed by existing methods while ensuring accurate data capture, compatibility with medical environments, and patient safety.

Objective of the Invention: The primary objective of the present invention is to provide an advanced EEG electrode system that overcomes the limitations of conventional EEG monitoring methods. This invention aims to offer a minimally invasive, adaptable, and comprehensive solution for monitoring brain activity in clinical settings, especially during neurosurgical procedures and other critical medical interventions. The proposed system is designed to provide accurate and reliable data capture, seamless integration with existing clinical monitoring platforms, and compatibility with a range of scenarios, thereby enhancing patient care and medical outcomes.

Brief Summary of the Invention: The disclosed invention presents an innovative in-the-ear EEG electrode system that addresses the challenges associated with traditional EEG monitoring techniques. This system incorporates both wireless and wired connectivity options, ensuring compatibility with diverse clinical environments and monitoring platforms. At its core, the system's eartips are constructed from a number of methods which include but are not limited to n-doped silicon or alternative conductive rubber-based materials, designed to efficiently detect and transmit neurological signals when positioned within the ear canal. By seamlessly interfacing with Multiparameter Monitors and other patient vital displays commonly used in clinical settings, the system facilitates real-time visualization of captured neurological data.

Key features of the invention include its ability to capture EEG data across various frequency bands, identify Event-Related Potentials (ERPs), and relay specific activities of the temporal lobes. The system is engineered to minimize interference from facial and scalp muscles, and it supports ambulatory use, making it suitable for dynamic clinical environments. Advanced signal processing techniques are integrated to filter out non-cerebral electrical activities, enhancing the accuracy of captured data. The invention aims to provide consistent and reliable neural activity monitoring, making it well-suited for applications demanding precise and continuous vital sign monitoring, particularly in intracranial procedures and similar specialized medical scenarios.

Technical Terminology and Concepts: Electroencephalogram (EEG): A screening and diagnostic method employed to measure and record electrical activity generated by the brain. This is done using electrodes placed on the scalp, which capture fluctuations in voltage resulting from ionic current flows within the neurons of the brain.

Electrodes: Conductive devices or mediums designed to detect electrical signals. In the context of the present invention, these are integrated into earbuds to facilitate non-invasive capture of EEG data.

EEG-Integrated Earbuds: A novel integration of EEG technology into earbuds. This involves the incorporation of electrodes within the earbuds' structure, enabling the continuous capture of EEG data while performing the primary function of audio delivery.

Alpha, Beta, Theta, Delta, and Gamma Waves: Different classifications of brainwaves based on their frequencies. Each type is associated with specific mental states or functions and can provide insights into brain health and activity.

EEG Amplifier: A component responsible for enhancing the electrical signals captured by EEG sensors. It ensures that the signals are of sufficient strength and clarity for subsequent processing and analysis.

Amplitude: In the context of EEG, amplitude refers to the magnitude of the electrical signal or brainwave at any given point in time.

Frequency: Represents the number of oscillations or cycles of a wave per unit of time. In EEG, it helps in categorizing brainwaves into types like Alpha, Beta, etc. Shape and Duration: These are graphical representations and lengths of time, respectively, for specific brainwaves or patterns. Variations in these parameters can offer insights into potential neurological anomalies or states of consciousness.

Multiparameter Monitor: A medical device used in clinical settings to continuously monitor patient vital signs. Some vitals that are currently measured are heart rate, electrocardiograms, blood pressure, respiratory rate, oxygen saturation, temperature, and more. The monitors mainly consist of a central display and multiple input components to attach wires and cables for different sensors.

### SUMMARY

In the realms of neurosurgery and intricate clinical procedures, obtaining precise and uninterrupted vitals through Electroencephalography (EEG) is paramount. However, existing EEG methodologies often present significant limitations and challenges. Conventional scalp EEG electrodes, while minimally invasive, are impractical to use during intracranial operations. On the other hand, alternative invasive EEG techniques, while providing deeper and potentially clearer data, come with associated risks, including the possibility of epidural hematomas and infections. Consequently, there is a need for an efficient, non-invasive system that can be employed across a variety of surgical and clinical scenarios without the drawbacks that current methods entail.

The invention presents an in-the-ear Electroencephalography electrode designed to address the limitations of current EEG monitoring techniques. This electrode system offers a dual connectivity framework, compatible with both wireless and wired configurations, providing adaptability to diverse clinical environments. The core functionality of the system lies in its eartip, which can be fabricated from n-doped silicon or an alternative conductive rubber-based material. This material selection ensures accurate and efficient detection of neurological signals when positioned within the ear canal. Moreover, the system has been engineered to interface seamlessly with various monitoring platforms, including the Multiparameter Monitor, facilitating the real-time visualization of neurological data in a variety of clinical settings.

The in-the-ear EEG electrode system captures and analyzes EEG data across multiple frequency bands, delineating brain activity states from delta to gamma. The system also identifies Event-Related Potentials (ERPs), specific brain responses synchronized to events such as sensory stimuli or motor actions. The electrode is positioned near the temporal lobes, recording activity crucial for auditory processing and epilepsy monitoring. Designed to be resilient against common artifacts and noise, the system minimizes interference from scalp and facial muscles. Its configuration supports ambulatory use, providing a solution to limitations found in conventional EEG systems. The in-ear placement seeks to minimize data variances attributed to individual differences in skull and brain anatomy. Advanced signal processing techniques are integrated to filter non-cerebral electrical activities, enhancing the accuracy of the captured data. The system's design accommodates prolonged monitoring in diverse settings without compromising data integrity.

The invention is an in-the-car EEG electrode system, designed to seamlessly connect, either wirelessly or through a wire, to a Multiparameter Monitor or other patient vital displays commonly utilized in Operating Rooms and Clinical Settings. At the core of the device are cartips constructed from n-doped electrically conductive silicon or alternative flexible and soft conductive rubber-based materials. When positioned within the ear canal, these earbuds serve as active electrodes, detecting and transmitting brain activity. This system provides the capacity to discern EEG data across designated frequency bands, capture Event-Related Potentials (ERPs), and relay specific activities of the temporal lobes. Its design reduces artifacts and extraneous noise, minimizing disturbances arising from facial and scalp muscles. Its compatibility with ambulatory use positions it as a viable tool in dynamic settings, addressing some challenges posed by traditional EEG systems. The electrode's design and materials aim to achieve consistent readings, mitigating discrepancies from individual anatomical differences. Integrated advanced signal processing techniques further refine the captured data, suppressing non-cerebral electrical activities to maintain accuracy. The overall design facilitates continuous, prolonged monitoring, ensuring that the data remains representative of the underlying neural activity.

The in-the-car EEG electrode system is primarily devised for use in operating rooms and clinical settings. Its main function is to enable continuous monitoring of brain activity through non-invasive means, particularly during surgical procedures, including intracranial operations. By capturing EEG data across designated frequency bands, the system provides real-time insights into various brain states, such as delta and gamma. The electrode's ability to detect Event-Related Potentials (ERPs) makes it valuable for identifying specific brain responses tied to particular events, such as sensory stimuli. The system's proximity to the temporal lobes positions it as a reliable tool for tracking activities linked to auditory processing or neurological conditions such as epilepsy. The design reduces interference from artifacts and noise, minimizing disturbances in facial and scalp muscles. Furthermore, the electrode's adaptability for ambulatory use ensures its practicality in more dynamic, real-world settings, presenting a solution to the limitations often associated with conventional EEG systems. The combination of advanced signal processing techniques ensures the minimization of non-cerebral electrical interference, thereby bolstering the accuracy of the recorded data, making it a preferred choice for applications demanding precise and consistent neural activity monitoring.

### BRIEF DESCRIPTIONS OF THE DRAWINGS

In the following portion relating to the detailed description, the embodiments of the present disclosure will be explained more in detail with reference to the example figures of the proposed invention shown in the drawings, which:

FIG. 1 depicts a side-view perspective of an individual using the n-doped silicon electrode in an EEG earbud system during a surgical operation.

FIG. 2 represents a front-view perspective of the presented invention in a wired EEG earbud system.

FIG. 3 portrays the entirety of the n-doped silicon electrode in an EEG earbud system and the attached chord for the Multiparameter Monitor.

FIG. 4 displays a flowchart that describes the processes that take place which allow for the invention to capture patient EEG data and display them on a multiparameter vitals monitor.

### DETAILED DESCRIPTION

This system is comprised of two primary components: the in-the-ear electrodes and their integration with wireless and wired connectivity to a multiparameter monitor or related devices.

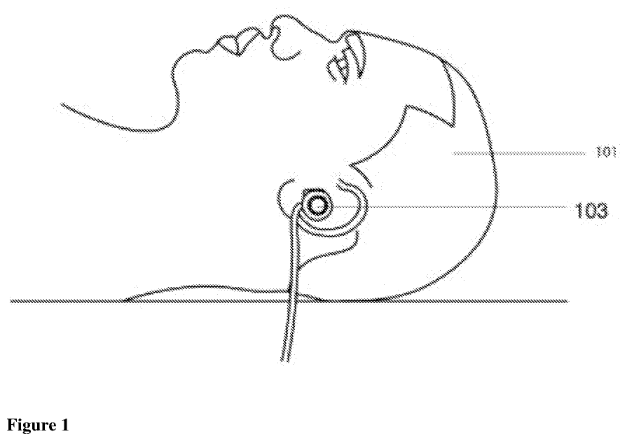

FIG. 1 provides a comprehensive lateral portrayal, illustrating the operational dynamics of the EEG earbud system within a surgical context. Reference number 101 represents a patient in a cerebral surgical procedure utilizing the in-ear electroencephalography (EEG) electrode system. This congruence is particularly relevant within open brain surgery and other intense clinical settings. The system seamlessly transmits crucial brain signal information to the surgeon. The primary objective of this system is to monitor and analyze cerebral activity, or detect EEG signals from various regions of the brain during medical procedures, thereby facilitating real-time insights for improved clinical outcomes.

Adjacent to the patient's ears is the earbud unit's core body 103. This grounding electrode body is designed to establish direct contact with the user's ears. This structural element houses critical aspects of the EEG earbud system, such as wiring, circuitry, and battery storage. This electrode also mitigates external noise, subsequently heightening the precision of EEG signal transmission while bolstering the operational efficiency of the surgical unit. The electrode system features wired and wireless configurations, ensuring adaptability to a wide spectrum of monitoring platforms.

Earbuds crafted from n-doped silicon and conductive rubber-based materials capture EEG signals from different brain regions, providing the surgeon with real-time awareness of cerebral activity during open brain surgery. While conventional EEG technology requires direct brain electrode placement, these earbuds are non-invasive, as they are strategically positioned within the ear canal to detect neurological signals. This scenario depicted in FIG. 1 is only one of numerous ways these earbuds can be used in clinical and surgical settings.

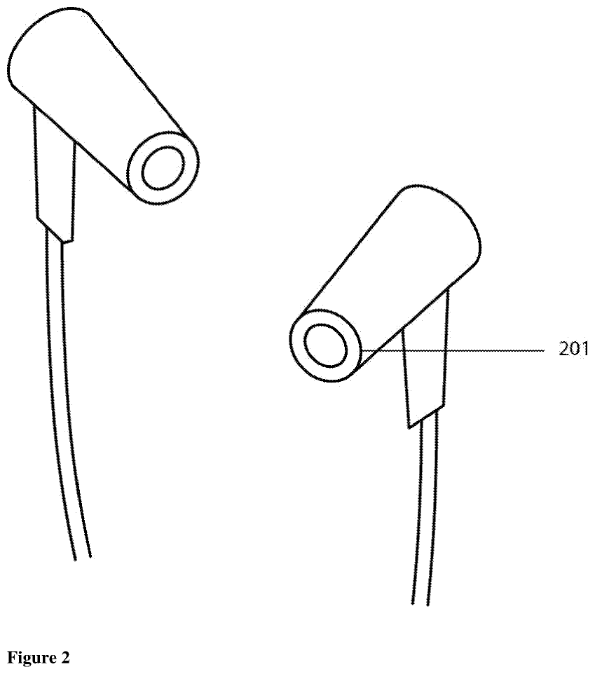

FIG. 2 depicts a front-view perspective of the in-the-ear EEG electrode system in a wired configuration. FIG. 2 depicts the specialized eartips constructed from n-doped electrically conductive rubber-based materials. These eartips, as illustrated by reference number 201, serve as active electrodes that capture and transmit brainwave signals when positioned within the ear canal. N-doped silicon is a biocompatible semiconductor material, ensuring precise signal capture while maintaining patient comfort. Conductive rubber-based materials are flexible and soft, making them suitable for conforming to the shape of the ear canal. The eartip's close proximity to the temporal lobes ensures an accurate recording of activities related to auditory processing and neurological responses. The diagram showcases the electrodes that are connected to a wiring system that leads to the Multiparameter Monitor or other patient vital displays.

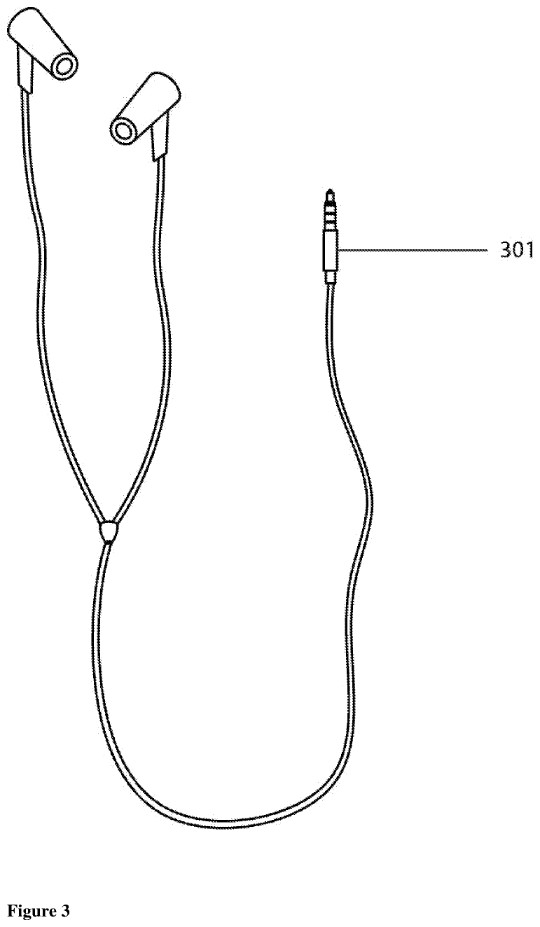

The wired configuration depicted in FIG. 2 offers important implications for the usage and engineering of the in-the-ear EEG electrode system in neurological and clinical settings. An important characteristic of the invention that allows it to be utilized in clinical settings is its connection jack 301 as depicted in FIG. 3. This connection jack is necessary in the case of wired connection of the invention. The cable of the wired device ends in a connector that allows for connection to a variety of devices meant for vitals sign monitoring. Connection of this connector to the device allows for transfer and display of data captured by the device in the case of a wired connection.

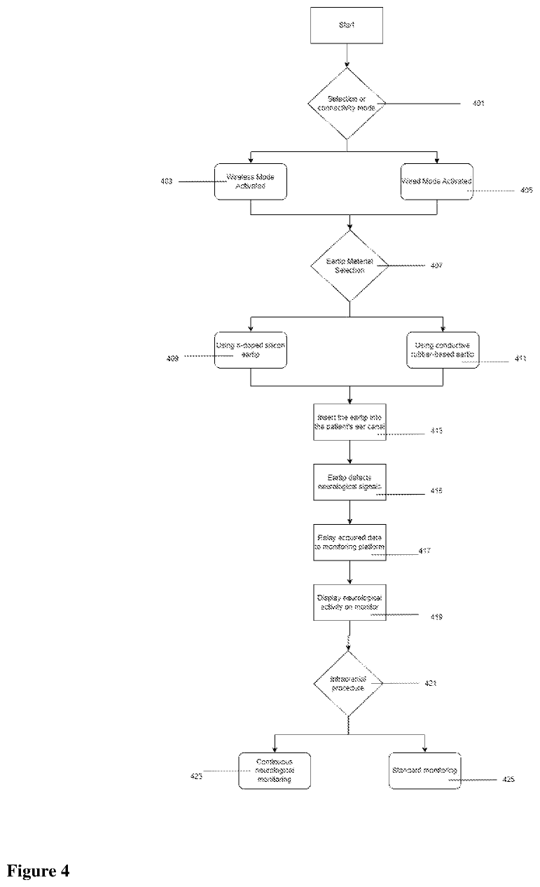

FIG. 4 illustrates the methodology employed in the development and integration of the preferred EEG wired earbud system into a multiparameter monitor. The process commences with the selection of the appropriate connectivity mode 401. Here, the options encompass two distinct configurations: wireless mode 403 and wired mode 405. The selection hinges on an evaluation of clinical and procedural criteria to ensure seamless integration.

Subsequently, the 407 task of eartip material selection occurs, aimed at optimizing electrical conductivity and detection of EEG signals into the patient monitoring system. This selection process offers two distinct choices: 409 n-doped electrically conductive silicone or 411 alternative conductive rubber-based silicone. The selection between these materials is contingent on patient compatibility and precise monitor requirements, allowing an efficient EEG signal acquisition process.

Following the selection of the material, the silicone eartip, integrated with the earbud apparatus, is positioned within the ear canal. During medical procedures that require the proposed invention, the eartip adeptly detects brain oscillation waves from various regions of the brain. Furthermore, the present invention directs this acquired data to the corresponding circuitry.

The transition of this neurological data 417 onto the monitoring system is a crucial step in the process. It is essential to acknowledge that the method of data transmission is contingent upon the type of connectivity previously chosen in 401. Upon successful transmission, the 419 EEG waves are displayed on the monitor, allowing for comprehensive monitoring and analysis.

Lastly, a decision point arises concerning the continuity of 421 intracranial procedure monitoring. In instances where an intracranial operation requires ongoing display of EEG waves in assessing brain health, 423 a course of continuous monitoring becomes imperative. However, if the procedure does not fall under the category of intracranial operations, the monitor may 425 continue standard monitoring.

1. An electrode system for monitoring brain activity, comprising:
a. An eartip constructed by a number of possible means, including but not limited to the use of n-doped silicon, conductive rubber based material, or silver covered eartip;
b. Said eartip configured to be positioned within an ear canal;
c. A wireless connectivity module integrated into said eartip, enabling wireless transmission of neurological signals;
d. A wired connectivity interface integrated into said eartip, enabling direct wired connection to monitoring systems;
e. Wherein said electrode system provide dual connectivity options for interfacing with diverse clinical monitoring platforms.

2. The electrode system of claim 1, wherein the n-doped silicone or conductive rubber-based material of the eartip is selected to ensure efficient and accurate detection of neurological signals when positioned within the ear canal.

3. The electrode system of claim 1, further compromising:
a. Compatibility with Multiparameter Monitor and other patient vital display commonly found in clinical environments;
b. Wherein the electrode system facilitates real-time visualization of captured neurological data on said monitoring displays.

4. The electrode system of claim 1, wherein:
a. The electrode system is particularly suitable for monitoring brain activity during intracranial operations;
b. The electrode system provides a mechanism to monitor and relay brain activity data to clinical monitoring systems, enhancing patient safety and surgical outcomes.

5. The electrode system of claim 1, wherein:
a. The electrode system's dual connectivity framework automatically adapts to wireless or wired configuration based on the monitoring platform's requirements;
b. The electrode system provides seamless data transmission regardless of the chosen connectivity mode.

6. The electrode system of claim 1, wherein:
a. The electrode system's dual connectivity framework automatically adapts to wireless or wired configurations based on the monitoring platform's requirements;
b. The electrode system provides seamless data transmission regardless of chosen connectivity mode.

7. An electrode system of claim 1, wherein the electrode system enables simultaneous monitoring of brain activity and vital signs, enhancing patient care in clinical settings.

8. An electrode system of claim 1, wherein the system is cross-compatible with existing electroencephalography systems, enabling seamless integration into clinical practices.

9. An electrode system as claimed in claim 1, further compromising:
a. Compatibility with both clinical and emergency medical service (EMS) monitoring platforms;
b. The electrode system provides dual connectivity options for interfacing with diverse monitoring platforms, enabling real-time visualization of neurological data during medical interventions, including EMS scenarios.
